(19) United States
(12) Patent Application Publication
(10) Pub. No.: US 2014/0356423 A1
(43) Pub. Date: Dec. 4, 2014

(54) FORMULATIONS OF LYCOPENE IN COMBINATION WITH BISPHOSPHONATES BONE RESORPTION INHIBITORS

(71) Applicant: Isaac A. Angres, North Potomac, MD (US)

(72) Inventor: Isaac A. Angres, North Potomac, MD (US)

(21) Appl. No.: 13/903,999

(22) Filed: May 28, 2013

(51) Int. Cl. A61K 31/66; A61K 31/01; A61K 9/48

(52) U.S. Cl. CPC A61K 31/66; A61K 9/48; A61K 31/01
USPC 424/456; 514/103; 514/762

(57) ABSTRACT

The present invention provides a pharmaceutical formulation suitable for filling softgel capsules comprising: (a) from about 1% to about 90% by weight of a bisphosphonic acid or a pharmaceutically acceptable salt; (b) from 1% to about 99% by weight of lycopene and (c) from about 40% to about 80% by weight of a liquid carrier comprising 50% to 80% by weight polyethylene glycol; 5% to 15% by weight of glycerin; and 5% to 20% by weight water. The invention also describes a method for preparing alendronate or its pharmaceutical acceptable salts in encapsulated therapeutic dosage form in combination with lycopene.

This application claims the priority benefit under 35 U.S.C. section 119 of U.S. Provisional Patent Application No. 61/651,639 entitled “Bisphosphonates Bone Resorption Inhibitors” filed on May 25, 2012; which is in its entirety herein incorporated by reference.

### FIELD OF THE INVENTION

The present invention relates to a carriers and compositions for pharmaceutically active ingredients and dosage forms containing the carrier composition and the active ingredient. The invention further relates to pharmaceutical solutions suitable for encapsulation in soft gelatin capsules. More particularly, the invention relates to pharmaceutically acceptable solvent systems capable of producing a solution of a medicament, such as a bone resorption inhibitor in combination with lycopene, for use in a soft gelatin capsule. This invention also relates to compositions containing bisphosphonates and lycopene for filling softgel capsules and their pharmaceutical use of in the treatment of conditions of abnormally increased bone turnover, such as osteoporosis.

The invention is also directed to compositions and methods for use in limiting undesired bone loss in a vertebrate at risk of such bone loss, in treating conditions that are characterized by undesired bone loss or by the need for bone growth, in treating fractures, and in treating cartilage disorders. More specifically, the invention concerns the use of specific bisphosphonate formulations in combination with lycopene for use in soft gel capsules.

The present invention also relates to oral compositions and methods for inhibiting bone resorption in a mammal. The compositions useful herein comprise the combination of a pharmaceutically effective amount of a bisphosphonate or a pharmaceutically-acceptable salt thereof in combination with lycopene in liquid vehicle that provides better bioavailability.

This invention is also concerned with a novel method for the prevention and/or treatment of diseases involving calcium or phosphate metabolism. In particular it is concerned with the prevention and/or treatment of diseases involving bone resorption, especially osteoporosis, Paget's disease, malignant hypercalcemia, periodontal disease, joint loosening and metastatic bone disease, by the administration of a bisphosphonic acid or a pharmaceutically acceptable salt thereof in combination with lycopene in novel carrier provided in a sofgel capsule.

This invention further relates to a novel oral pharmaceutical composition with improved bioavailability for treating osteoporosis while minimizing the occurrence of or potential for adverse gastrointestinal effects.

### BACKGROUND OF THE INVENTION AND DESCRIPTION OF THE PRIOR ART

Normal bones are living tissues undergoing constant resorption and redeposition of calcium, with the net effect of maintenance of a constant mineral balance. By virtue of the fact, that bone is not a static tissue, it is subject to constant breakdown and resynthesis in a complex process mediated by osteoblasts, which produce new bone, and osteoclasts, which destroy bone. Osteoclasts are multinucleated cells which are responsible for causing bone loss through a process known as bone resorption. Osteoclasts are actively motile cells that migrate along the surface of bone, and that can bind to bone and secrete acids and proteases causing a resorption of mineralized bone tissue. The activities of these cells are regulated by a large number of cytokines and growth factors, many of which have now been identified and cloned.

The process described above is commonly called “bone turnover”. In normal growing bones, the mineral deposition is in equilibrium with the mineral resorption, whereas in certain pathological conditions, bone resorption exceeds bone deposition, for instance due to malignancy or primary hyperparathyroidism, or in osteoporosis. In other pathological conditions the calcium deposition may take place in undesirable amounts and areas leading to e.g., heterotopic calcification, osteoarthritis, kidney or bladder stones, atherosclerosis, and Paget's disease which is a combination of an abnormal high bone resorption followed by an abnormal calcium deposition. Calcium-related disorders in general and osteoporosis in particular are a major public health problem in developed countries.

Osteoporosis is caused by a reduction in bone mineral density in mature bone and results in fractures after minimal trauma. It is the most common type of metabolic bone disease in the U.S., and the condition affects more than 25 million people. The disease causes more than 1.3 million fractures each year, including 500,000 spine, 250,000 hip and 240,000 wrist fractures annually. The disease is widespread and has a tremendous economic impact. Osteoporosis is a systemic skeletal disease characterized by a low bone mass and microarchitectural deterioration of bone tissue, with a consequent increase in bone fragility and susceptibility to fracture. The most common fractures occur in the vertebrae, distal radius and hip. Osteoporotic fractures are a major cause of morbidity and mortality in the elderly population. As many as 70% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment. An estimated one-third of the female population over age 65 will have vertebral fractures, caused in part by osteoporosis. Moreover, hip fractures are likely to occur in about one in every three woman and one in every six men by extreme old age.

Two distinct phases of bone loss have been identified. One is a slow, age-related process that occurs in both genders and begins at about age 35. This phase has a similar rate in both genders and results in losses of similar amounts of cortical and cancellous bone. Cortical bone predominates in the appendicular skeleton while cancellous bone is concentrated in the axial skeleton, particularly the vertebrae, as well as in the ends of long bones. Osteoporosis caused by age-related bone loss is known as Type II osteoporosis.

The other type of bone loss is accelerated, seen in postmenopausal women and is caused by estrogen deficiency. This phase results in a disproportionate loss of cancellous bone. Osteoporosis due to estrogen depletion is known as Type I osteoporosis. The main clinical manifestations of Type I osteoporosis are vertebral, hip and forearm fractures. The skeletal sites of these manifestations contain large amounts of trabecular bone. Bone turnover is usually high in Type I osteoporosis. Bone resorption is increased but there is inadequate compensatory bone formation. Osteoporosis has also been related to corticosteroid use, immobilization or extended bed rest, alcoholism, diabetes, gonadotoxic chemotherapy, hyperprolactinemia, anorexia nervosa, primary and secondary amenorrhea, transplant immunosuppression, and oophorectomy.

It is also known that Bisphosphonates have been widely used to inhibit osteoclast activity in a variety of both benign and malignant diseases in which bone resorption is increased. Bisphosphonates are non-hormonal treatments for osteoporosis which work by “switching off” the cells that break down bone, allowing the bone building cells to work more efficiently. Thus bisphosphonates have recently become available for long-term treatment of patients with Multiple Myeloma. Bisphosphonates are carbon-substituted pyrophosphate analogues that include potent inhibitors of bone resorption, such as alendronate (4-amino-1-hydroxybutylidene-1,1-biphosphonic acid) (Sato et al. (1991) J. Clin. Invest. 88, 2095-2105). These pyrophosphate analogs not only reduce the occurrence of skeletal related events but they also provide patients with clinical benefit and improve survival. Bisphosphonates are able to prevent bone resorption in vivo and the therapeutic efficacy of bisphosphonates has been demonstrated in the treatment of Paget's disease of bone, tumour-induced hypercalcemia and, more recently, bone metastasis and multiple myeloma (for review see Fleisch H, Bisphosphonates Clinical. In Bisphosphonates in Bone Disease. From the Laboratory to the Patient. Eds: The Parthenon Publishing Group, NewYork/London pp 68-163, 1997).

At this time, the mechanisms by which bisphosphonates inhibit bone resorption are not well understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit both the activation and the activity of osteoclasts.

In addition bisphosphonates have been proposed for use in the treatment of osteoporosis. For example, U.S. Pat. No. 4,812,304 discloses a method for treating or preventing osteoporosis in humans comprising administering to a subject afflicted with or at risk to osteoporosis a bone cell activating compound and a bone resorption inhibiting polyphosphonate according to a regime consisting of one or more cycles, whereby each cycle consists of: (a) a bone activating period of from about 1 day to about 5 days during which a bone cell activating amount of a bone cell activating compound is administered to said subject; followed by (b) a bone resorption inhibition period of from about 10 days to about 20 days during which ethane-1-hydroxy-1,1-diphosphonic acid, or a pharmaceutically acceptable salt or ester thereof, is administered daily to said subject in an amount from about 0.25 mg/kg/day to about 3.3 mg/kg/day; followed by (c) a rest period of from about 70 days to about 180 days during which the subject receives neither a bone cell activating compound nor a bone resorption inhibiting polyphosphonate.

Also, U.S. Pat. No. 4,761,406 teaches a method for treating osteoporosis, in humans or lower animals afflicted with or at risk of osteoporosis, comprising administering to said human or lower animal an effective amount of a bone resorption inhibiting polyphosphonate according to the following schedule: (a) a period of from about 1 day to about 90 days during which said bone resorption inhibiting polyphosphonate is administered daily in a limited amount, followed by (b) a rest period of from about 50 days to about 120 days, and (c) repeating (a) and (b) two or more times where a net increase in bone mass of said human or animal results.

The oral bioavailability of bisphosphonates (etidronate; clodronate; pamidronate; alendronate) in humans lies between 1% and 10% according to Lin (Bone 18, 75-85, 1996) and absorption is diminished when given with meals, especially in the presence of calcium. Therefore bisphosphonates should never be given at mealtime and never together with milk or diary products according to Fleisch (Bisphosphonates in bone disease, Stampli & Co., Bern p. 50, 1993 and references cited therein).

The oral bioavailability of alendronate has been studied by Gertz et al. (Clinical Pharmacology & Therapeutics, vol. 58, pp. 288-298, 1995). It was found that taking alendronate either 60 or 30 minutes before breakfast reduced bioavailability by 40% relative to a 2-hour wait before a meal. Taking alendronate either concurrently with or 2 hours after breakfast drastically (>85%) impaired availability. A practical dosing recommendation, derived from these findings was that patients should take the drug with water after an overnight fast and at least 30 min before any other food or beverage. Additionally, Alendronate has been approved by various regulatory agencies, including the Food and Drug Administration in the United States as an oral osteoporosis treatment in post menopausal women. The currently marketed formulation is a tablet, and the patient is instructed to take the tablet with a full glass of water in the morning, at least a half hour prior to eating or drinking. However, certain side effects, including esophageal irritation and erosion have been reported if the tablet was not taken with enough water, or if the patient did not remain in an upright position for approximately one-half hour after taking the medication. Without being limited by theory, it is believed that the potential gastrointestinal effects of oral bisphosphonates is related to the induction of apoptosis, i.e. programmed death, of the cells of the epithelial lining of the gastrointestinal tract.

Additionally, the potent antioxidant properties of lycopene, the strong evidence for the role of oxidative stress in bone health, and the limited reported studies on the effects of lycopene in bone cells in culture may have important implications for the beneficial role of lycopene in bone health.

Bone is a dynamic organ that undergoes continuous remodeling, the tightly regulated coupling between the resorption of old bone by osteoclasts and the formation of new bone by osteoblasts that is fundamental to normal bone physiology. Disturbances in bone remodeling lead to bone diseases. Oxidative stress, shown to control the function of both osteoclasts and osteoblasts, may contribute to the pathogenesis of skeletal system including the most prevalent metabolic disease, osteoporosis.

Very little work has been reported on the role of oxidative stress in osteoblasts. However, previous reports demonstrated that ROS is involved in osteoblast function. The effect of lycopene in osteoblasts showed that lycopene stimulated the proliferation of the osteoblast-like SaOS-2 cells. Lycopene is also known to stimulate alkaline phosphatase activity in the more mature cells grown in the presence of dexamethasone (SaOS+Dex cells), but inhibited or had no effect in younger cells grown in the absence of dexamethasone (SaOS-Dex cells), depending on the time of addition. It has been postulated that Lycopene acts as a potent antioxidant, inhibiting oxidative damage caused by the highly reactive oxygen species (ROS) produced intracellularly in SaOS-2 cells or it could involve cell cycling genes.

Although the mechanisms involved in the differentiation of osteoclasts and its ability to resorb bone are poorly understood, one theory suggests that ROS are involved in these processes. A number of studies revealed that ROS increase bone resorption. Others suggested that ROS may be involved in the regulation of osteoclast formation and osteoclast motility.

There is evidence to suggest that ROS-induced oxidative stress is associated with the pathogenesis of osteoporosis. Epidemiological studies presented evidence suggesting that certain antioxidants including vitamin C, E and beta-carotene may reduce the risk of osteoporosis and counteract the adverse effects on bone of the oxidative stress produced during strenuous exercise and in heavy smokers. Also, it appears that women with osteoporosis had markedly decreased plasma antioxidants.

Consequently, there is a need for pharmaceutical formulations comprising lycopene and bisphosphonates, such as alendronate, which reduces the above mentioned drawbacks and allows the patient to take the medicament more conveniently, e.g. together with food intake. Also, it would be desirable to develop a formulation which allows a bisphosphonate to be taken orally in combination with lycopene, yet avoids problems associated with prolonged contact between the medication and the esophagus.

### OBJECTS OF THE INVENTION

It is an object of the present invention to provide pharmaceutical compositions containing a bone resorption inhibitor and lycopene.

It is also an object of the present invention to provide softgel capsules containing bone resorption inhibitors and lycopene.

It is another object of the present invention to provide pharmaceutical formulations containing bisphosphonate bone resorption inhibitors and lycopene in a novel formulation useful for filling softgel capsules.

A still further object of the invention is to provide softgel capsules containing bisphosphonate bone resorption inhibitors and lycopene.

A further object of the invention is to provide softgel capsules containing bisphosphonate bone resorption inhibitors and lycopene having improved bioavailability.

An additional object of the present invention is to provide improved oral compositions for inhibiting bone resorption and the conditions associated therewith in a mammal, particularly wherein said mammal is a human.

A further object of the present invention to treat or prevent abnormal bone resorption in an osteoporotic mammal, preferably an osteoporotic human using bisphosphonate inhibitors in combination with lycopene formulated in softgel capsules.

It is an additional object of the invention to provide a method for treating an individual having a condition that is responsive to administration of a bisphosphonic acid compound and lycopene, by administering a dosage form as provided herein within the context of an effective dosing regimen.

Other objects and embodiments of the present invention will be discussed below. However, it is important to note that many additional embodiments of the present invention not described in this specification may nevertheless fall within the spirit and scope of the present invention and/or the claims.

### SUMMARY OF THE INVENTION

The present invention is directed to a pharmaceutical formulation comprising: (a) a therapeutically effective amount of a bone resorption inhibitor; and (b) lycopene.

The present invention is further directed to a pharmaceutical formulation suitable for filling softgel capsules comprising: (a) a therapeutically effective amount of a bone resorption inhibitor; (b) lycopene and (c) a solvent system comprising 50% to 85% by weight a polyethylene glycol; 5% to 15% by weight of glycerin and 5% to 20% by weight water.

The instant invention is also directed to a pharmaceutical soft gelatin capsule in unit dosage form with a filling comprising a therapeutically effective amount of a bisphosphonate; a therapeutically effective amount of lycopene and a solvent system comprising 50% to 85% by weight a polyethylene glycol; 5% to 15% by weight of glycerin and 5% to 20% by weight water.

The invention also provides a pharmaceutical formulation for oral administration having increased stability and bioavailability, comprising a soft gelatin capsule which essentially contains a therapeutically active amount of said bisphosphonate bone resorption inhibitor and lycopene dissolved in a composition comprising: 50% to 80% by weight polyethylene glycol; 5% to 15% by weight of glycerin; 5% to 20% by weight.

The present invention also features a liquid softgel fill formulation comprising: (a) from about 10% to about 90% by weight of a bisphosphonic acid or a pharmaceutically acceptable salt; (b) 10% to 90% by weight of lycopene and (c) a liquid carrier comprising 50% to 80% by weight polyethylene glycol; 5% to 15% by weight of glycerin; 5% to 20% by weight.

The invention also relates to an oral dosage form comprising effective amounts of at least one bisphosphonate, lycopene and a carrier comprising a mixture of polyethylene glycol, glycerine, and water.

The invention also relates to an oral dosage form comprising effective amounts of alendronate, lycopene and a carrier comprising a mixture of polyethylene glycol, glycerine, and water.

The present invention also describes a pharmaceutical formulation suitable for filling softgel capsules comprising: (a) a therapeutically effective amount of a bone resorption inhibitor; (b) a therapeutically effective amount of lycopene and (c) a solvent system comprising 50% to 85% by weight a polyethylene glycol; 5% to 15% by weight of glycerin and 5% to 20% by weight water wherein said polyethylene glycol is a mixture of a polyethylene glycol having an average molecular weight of 400 and a polyethylene glycol having an average molecular weight of 3400.

The invention is further directed to a process for the preparation of a fill solution for softgels containing an active ingredient selected from the group consisting of: 4-amino-1-hydroxybutylidene-1,1-bisphosphonic acid (alendronate); N,N-dimethyl-3-amino-1-hydroxypropylidene-1,1-bisphosphonic acid (mildronate, olpadronate); 1-hydroxy-3-(N-methyl-N-pentylamino)propylidene-1,1-bis-phosphonic acid (ibandronate); 1-hydroxy-2-(3-pyridyl)ethylidene-1,1-bisphosphonic acid (risedronate); 1-hydroxyethylidene-1,1-bisphosphonic acid (etidronate); 1-hydroxy-3-(1-pyrrolidinyl)propylidene-1,1-bisphosphonic acid; 1-hydroxy-2-(1-imidazolyl)etylidene-1,1-bisphosphonic acid (zoledronate); 1-hydroxy-2-(imidazo[1,2-a]pyridin-3-yl)ethylidene-1,1-bisphosphonic acid (minodronate); 1-(4-chlorophenylthio)-methylidene-1,1-bisphosphonic acid (tiludronate); 1-(cycloheptylamino)methylidene-1,1-bisphosphonic acid (cimadronate, incadronate); and 6-amino-1-hydroxyhexylidene-1,1-bisphosphonic acid (neridronate), or a pharmaceutically acceptable salt thereof; which process comprises: (1) milling said bisphosphonates to a particle size of about 50 to 100 μm; (2) mixing the bisphosphonate active ingredient with polyethylene glycol, glycerine and water and; (3) agitating the resulting solution until homogeneous at a temperature of from about 40° C. to about 65° C. After milling the bisphosphonate is mixed with lycopene. The combination is used in effective amounts to administer to a mammal.

The invention also features a method for preparing alendronate or its pharmaceutical acceptable salts in combination with lycopene in encapsulated therapeutic dosage form, comprising the steps of reducing the size of alendronate particles to an average size no larger than about 80 microns, then mixing the micronized particles of alendronate with lycopene and then with a solvent essentially consisting predominantly of polyethylene glycol of a molecular weight no greater than about 1000, and heating the mixture at a temperature of from about 40° C. to about 50° C. until the alendronate and lycopene are dissolved in the solvent, and then encapsulating therapeutic doses of the dissolved alendronate and lycopene in gelatin capsules soluble in water but insoluble in said solvent.

### DETAILED DESCRIPTION OF THE INVENTION

The present invention provides novel therapeutic formulations containing effective amounts of bisphosphonates and effective amounts of lycopene.

The present invention also provides a solvent system for preparing solutions of bisphosphonates pharmaceutical agents and lycopene wherein the prepared solutions are particularly suitable for softgel filling. The solvent system of the invention contains polyethylene glycol, a C₃-C₅ polydroxyl compound and water. The solvent system of the invention comprises 50% to 85% by weight of a polyethylene glycol; 5% to 15% by weight C₃-C₅ polydroxyl compound and 5% to 20% by weight water. A more preferred solvent system of the invention contains 50% to 85% by weight of a polyethylene glycol; 5% to 15% by weight of glycerine and 5% to 20% by weight water. A most preferred solvent system contains 50% to 85% of polyethylene glycol wherein the polyethylene glycol is a mixture of a polyethylene glycol having an average molecular weight from about 200-800, and a polyethylene glycol having an average molecular weight from about 1,000-10,000, 5% to 15% by weight of glycerine and 5% to 20% by weight water. A particularly most preferred solvent system contains 50% to 85% of polyethylene glycol wherein the polyethylene glycol is a mixture of a polyethylene glycol having an average molecular weight of about 400, and a polyethylene glycol having an average molecular weight of about 3400, 5% to 15% by weight of glycerine and 5% to 20% by weight water. The mixture of the two types of polyethylene glycols is typically 1% to 99% by weight of a polyethylene glycol having an average molecular weight from about 200-800, and 1% to 99% by weight of a polyethylene glycol having an average molecular weight from about 1,000-10,000. The more preferred mixture of polyethylene glycols contains 40% to 98% by weight of a polyethylene glycol having an average molecular weight from about 200-800, and 2% to 60% by weight of a polyethylene glycol having an average molecular weight from about 1,000-10,000. The most preferred mixture of polyethylene glycols contains 80% to 97% of a polyethylene glycol having an average molecular weight from about 200-800, and 3% to 20% by weight of a polyethylene glycol having an average molecular weight from about 1,000-10,000.

Polyethylene glycols generally are clear, viscous liquids or white solids which are soluble in water and many organic solvents. These polymers correspond to the general formula:

H(OCH₂CH₂)ₙOH

where n is greater than or equal to 4. Polyethylene glycols are described in G. M. Powell, III in Handbook of Water-Soluble Gums & Resins, R. L. Davidson, Ed. (McGraw-Hill, New York, 1980) pp. 18/1-18/31, this reference being incorporated herein by reference in its entirety. Polyethylene glycols, which are also known as “PEGs” or “polyoxyethylenes”, are designated by both their average molecular weight range and their average “n” value as in the above designated formula. For example, polyethylene glycol 400, which is also known by the CTFA designation, PEG-B, has an average molecular weight range from 380-420 and an average value of n between 8.2 and 9.1. See CTFA Cosmetic Ingredient Dictionary, Third Edition (1982), pp. 201-203; and The Merck Index, Tenth Edition, entry 7441, p. 1092 (1983), incorporated herein by reference.

The polyethylene glycols preferable herein are those which are liquids at room temperature or have a melting point slightly thereabove or are in powder form. Preferred are the polyethylene glycols having a molecular weight range from about 300 to about 4600 and corresponding n values from about 6 to about 104. Most preferred are the polyethylene glycols having a molecular weight range from about 400 to about 3400 and corresponding n values from about 8 to about 76. Liquid and low-melting polyethylene glycols are commercially available from Union Carbide (Danbury, Conn.) under the Carbowax® trademark. See “Carbowax® Polyethylene Glycols”, Union Carbide Technical Bulletin f-4772M-ICD 11/86-20M, this reference being incorporated herein by reference in its entirety.

The polyethylene glycols useful herein are those which are liquids at room temperature and those which are in powder form at room temperature. The ones that are liquid at room temperature are low molecular weight polyethylene glycols having an average molecular weight from about 200-800, while those that are in powder form are polyethylene glycols having an average molecular weight from about 1,000-10,000.

Depending on the bisphosphonate pharmaceutically active, varying amounts of polyethylene glycol may be employed to facilitate dissolution of the pharmaceutically active material. The same logic applies when combined with the lycopene.

The pharmaceutical agents suitable for use with the solvent system of this invention are bone resorption inhibitors and more in particular bisphosphonate bone resorption inhibitors in combination with lycopene. The bisphosphonic acid compounds may be in crystalline or amorphous form, and mixtures of bisphosphonic acids may be employed. The bisphosphonic acid may also be in the form of a pharmaceutically acceptable salt, ester, anhydride, carbamate, amide, hydrate, or other analog, derivative or prodrug, or a combination thereof (e.g., a sodium salt trihydrate, as in alendronate). Salts of the bisphosphonic acid compounds may be obtained commercially or can be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, amino acids, and the like, as well as inorganic acids, e.g., hydrochloric acid, hydrobromic acid, sulfuric acid, nitric acid, phosphoric acid, and the like. Basic salts of acid moieties, e.g., phosphonic acid groups, may be prepared using a pharmaceutically acceptable base. Salts formed with the phosphonic acid group include, but are not limited to, alkali metal salts, alkaline earth metal salts and organic base salts. For example, bases such as sodium hydroxide, potassium hydroxide, ammonium hydroxide, calcium hydroxide, magnesium hydroxide, trimethylamine, lysine, arginine, triethanolamine, and the like, may be used. Preparation of esters involves functionalization of hydroxyl and/or carboxyl groups which may be present. These esters are typically acyl-substituted derivatives of free alcohol groups, i.e., moieties which are derived from carboxylic acids of the formula RCOOH where R is alkyl, and preferably is lower alkyl. Pharmaceutically acceptable esters may be prepared using methods known to those skilled in the art and/or described in the pertinent literature. Anhydrides, carbamates, amides, hydrates, and other analogs, derivatives and prodrugs can be readily prepared as well, using conventional means, and incorporated into the present formulations.

The preferred biphosphonates are selected from the group consisting of: 4-amino-1-hydroxybutylidene-1,1-bisphosphonic acid (alendronate); N,N-dimethyl-3-amino-1-hydroxypropylidene-1,1-bisphosphonic acid (mildronate, olpadronate); 1-hydroxy-3-(N-methyl-N-pentylamino)propylidene-1,1-bis-phosphonic acid (ibandronate); 1-hydroxy-2-(3-pyridyl)-ethylidene-1,1-bisphosphonic acid(risedronate); 1-hydroxyethylidene-1,1-bisphosphonic acid (etidronate); 1-hydroxy-3-(1-pyrrolidinyl)propylidene-1,1-bisphosphonic acid; 1-hydroxy-2-(1-imidazolyl)etylidene-1,1-bisphosphonic acid (zoledronate); 1-hydroxy-2-(imidazo[1,2-a]pyridin-3-yl)ethylidene-1,1-bisphosphonic acid (minodronate); 1-(4-chlorophenylthio)methylidene-1,1-bisphosphonic acid (tiludronate); 1-(cycloheptylamino)methylidene-1,1-bisphosphonic acid (cimadronate, incadronate); and 6-amino-1-hydroxyhexylidene-1,1-bisphosphonic acid (neridronate), and pharmaceutically acceptable salts thereof.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium. A very preferred group of pharmaceutically acceptable salts is characterized by having one acidic hydrogen and one pharmaceutically acceptable cation, especially sodium, in each of the phosphonic acid groups.

Methods for the preparation of bisphosphonic acids may be found in, e.g., U.S. Pat. No. 3,962,432; U.S. Pat. No. 4,054,598; U.S. Pat. No. 4,267,108; U.S. Pat. No. 4,327,039; U.S. Pat. No. 4,407,761; U.S. Pat. No. 4,621,077; U.S. Pat. No. 4,624,947; U.S. Pat. No. 4,746,654; and U.S. Pat. No. 4,922,077. More in particular, methods for the preparation of 4-amino-1-hydroxybutylidene-1,1-bisphosphonic acid and 4-amino-1-hydroxy-butylidene-1,1-bisphosphonic acid monosodium salt trihydrate may be found in U.S. Pat. No. 4,407,761 and U.S. Pat. No. 4,922,077, respectively.

In the instant invention, it is preferred that the bisphosphonic acid is 4-amino-1-hydroxybutylidene-1,1-bisphosphonic acid. It is even more preferred that the bisphosphonic acid is a sodium salt of 4-amino-1-hydroxybutylidene-1,1-bisphosphonic acid, in particular, 4-amino-1-hydroxybutylidene-1,1-bisphosphonic acid monosodium salt trihydrate.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis.

For once-weekly dosing, an oral unit dosage comprises from about 17.5 mg to about 70 mg of the alendronate compound, on an alendronic acid active weight basis. Examples of weekly oral dosages include a unit dosage which is useful for osteoporosis prevention comprising about 35 mg of the alendronate compound, and a unit dosage which is useful for treating osteoporosis comprising about 70 mg of the alendronate compound.

The lycopene is provided in dosages ranging from 10-100 mg when combined with the bisphosphonates.

Other components which can be incorporated into the compositions of the instant invention include colorings, flavorings, preservatives, lubricants, flow-enhancers, filling aids, antioxidants, essences, and other aesthetically pleasing components.

Prior to making the solutions for encapsulation the bisphosphonate is micronized. Micronization of the bisphosphonate ie., alendronate, the first step in the process of this invention, involves any method for reducing the solid alendronate to an average particle size of 80 microns or less. Hammer milling, ball milling, air impaction, or any commonly known comminution technique may be used for that purpose. To eliminate even a small proportion of relatively large particles (i.e., particles larger than 85 or 90 microns in size), the finely divided alendronate should be passed through a screen at least as fine as 195 mesh, and preferably 175 mesh.

The alendronate so micronized is combined with the lycopene and then dissolved in a liquid carrier consisting predominately of a polyethylene glycol having an average molecular weight from about 200-800. To hasten dissolution of the alendronate in the polyethylene glycol vehicle, moderate heating to temperatures within the range of 50° C.-70° C. may be employed; however, temperatures substantially above 100° C. should be avoided because of the danger of decomposing the drug. Following dissolution of the micronized alendronate in combination with the lycopene at a concentration level of at least 50 milligrams per milliliter (mg per ml), and preferably at a concentration of 60 (or more) mg per ml, the solution is cooled to a temperature of 35° C. or less and is sealed in soft gelatin capsules utilizing standard (and well known) encapsulating equipment and procedures. In effect, the liquid alendronate/lycopene solution is injected into a soft gelatin capsule as that capsule is being formed, the final step consisting of the sealing of the capsule at the point of fluid entry.

To hasten dissolution of the micronized alendronate and lycopene in the liquid vehicle, the vehicle may contain small amounts (no more than about 25 percent, and preferably more than 15 percent, by weight) of other ingredients such as glycerin, propylene glycol, or relatively high molecular weight polyethylene glycol. Thus, blends of low molecular weight (400) polyethylene glycol with 7 percent glycerin, or 5 percent propylene glycol, have been found effective. A particularly effective blend consists predominately of polyethylene glycol (400) with a minor amount (under 5 percent) of high molecular weight (4000) polyethylene glycol, since such a blend is essentially thixotropic. At room temperature it is a semi-solid but, upon agitation or moderate heating (under 35° C.) it becomes a liquid. Therefore, such a blend may be easily sealed within soft gelatin capsules by means of customary filling equipment and techniques and, after the capsules have cooled, the solution becomes a semi-solid to render the capsules virtually leakproof during normal storage and handling at room temperature or below. Under such temperature conditions, even if the gelatin casing of the capsule should somehow become damaged, or an imperfection should cause a small opening to develop, the contents would remain in place.

The compositions in accordance with the invention will generally be prepared by dissolving the bisphosphonate and the lycopene or its pharmaceutically acceptable salt in a mixture of polyethylene glycol, glycerine and water and for this purpose it is generally preferred that the amount of water be sufficient to insure solution of the active ingredient in the water/PEG/glycerine mixture.

The solubilized pharmaceutical compositions of the present invention can be encapsulated within any conventional soft gelatin shell that is capable of substantially containing the composition for a reasonable period of time. The soft gelatin shells of the instant invention can be prepared by combining appropriate amounts of gelatin, water, plasticizer, and any optional components in a suitable vessel and agitating and/or stirring while heating to about 65° C. until a uniform solution is obtained. This soft gelatin shell preparation can then be used for encapsulating the desired quantity of the solubilized fill composition employing standard encapsulation methodology to produce one-piece, hermetically-sealed, soft gelatin capsules.

In a nut shell, the formation of soft gelatin capsules is carried out in a stamping process wherein the capsule wall is assembled from two gelatin halves which are stamped out of a gelatin band and then molded. Preferably, there is utilized the Scherer process operating under the rotary die method. Herein two endless gelatin bands run against two adjacent and mutually counter-rotating molding rollers. While the gelatin bands are being pressed into the molded form and so create the capsule halves, the flowable filler is provided into the thus formed capsule through an exact dosing wedge. There follows the sealing together of the capsule halves, their stamping out, a wash procedure for the freeing of attached oil, a rotational dryer step as well as an adjacent shelf drying.

More specifically, the fill formulation of the instant invention is encapsulated into one-piece gelatin sheath or shell that includes a plasticizer to control the softness and flexibility of the sheath, water, and optionally, other additives, such as flavorants, colorants, opacifiers, etc. The softgel capsules may be produced in a known manner with a rotary die process in which a molten mass of a gelatin sheath formulation is fed from a reservoir onto drums to form two spaced sheets or ribbons of gelatin in a semi-molten state. These ribbons are fed around rollers and brought together at a convergent angle into the nip of a pair of roller dies that include opposed die cavities. A fill formulation to be encapsulated is fed into the wedge-shaped joinder of the ribbons.

The gelatin ribbons are continuously conveyed between the dies, with portions of the fill formulation being trapped between the sheets inside the die cavities. The sheets are then pressed together, and severed around each die so that opposed edges of the sheets flow together to form a continuous gelatin sheath around the entrapped medicament. The part of the gelatin sheet that is severed from the segments forming the capsules is then collected for recycling, and the soft capsules are dried.

Various sheath formulations known in the prior art may be used to encapsulate the fill formulations of the present invention. For example, suitable sheath formulations may include from about 30 to about 50% by weight gelatin; at least 18% by weight, and preferably up to about 40% by weight, of a plasticizer; and from about 20 to about 50% by weight water. These formulations, when formed into capsules and dried, will result in capsule sheaths comprised of from about 40 to about 75% by weight gelatin; from about 18% to about 40% by weight plasticizer; and from about 5 to about 15% by weight water. Another preferred capsule contains gelatin plasticised with from about 3 to 15% of weight of glycerin preferably about 6% by weight, and from 12.5 to 15% by weight of sorbitol, preferably about 14% of sorbitol, the percentages being based on the total weight of gelatin, glycerin and sorbitol.

The gelatin will normally have a bloom in the range of from about 140 to about 280, and may be Type A or B gelatins or a mixture thereof. Limed bone, acid bone, fish and/or pig skin gelatins may be used.

The gelatin capsules are formed into the desired shape and size so that they can be readily swallowed. The soft gelatin capsules of the instant invention are of a suitable size for easy swallowing and typically contain from about 10 mg to about 2000 mg of the solubilized pharmaceutical active composition. Soft gelatin capsules and encapsulation methods are described in P. K. Wilkinson et al., “Softgels: Manufacturing Considerations”, Drugs and the Pharmaceutical Sciences, 41 (Specialized Drug Delivery Systems), P. Tyle, Ed. (Marcel Dekker, Inc., New York, 1990) pp. 409-449; F. S. Hom et al., “Capsules, Soft” Encyclopedia of Pharmaceutical Technology, vol. 2, J. Swarbrick and J. C. Boylan, eds. (Marcel Dekker, Inc., New York, 1990) pp. 269-284; M. S. Patel et al., “Advances in Softgel Formulation Technology”, Manufacturing Chemist, vol. 60, no. 7, pp. 26-28 (July 1989); M. S. Patel et al., “Softgel Technology”, Manufacturing Chemist, vol. 60, no. 8, pp. 47-49 (August 1989); R. F. Jimerson, “Softgel (Soft Gelatin Capsule) Update”, Drug Development and Industrial Pharmacy (Interphex '86 Conference), vol. 12, no. 8 & 9, pp. 1133-1144 (1986); and W. R. Ebert, “Soft Elastic Gelatin Capsules: A Unique Dosage Form”, Pharmaceutical Technology, vol. 1, no. 5, pp. 44-50 (1977); these references are incorporated by reference herein in their entirety. The resulting soft gelatin capsule is soluble in water and in gastrointestinal fluids. Upon swallowing the capsule, the gelatin shell rapidly dissolves or ruptures in the gastrointestinal tract thereby introducing the pharmaceutical actives into the physiological system.

The novel drug dosage forms are to be administered orally to a mammalian individual and can be used to administer a bisphosphonic acid compound in combination with lycopene as an active agent with minimal, if any, side effects. In accordance with the present invention, administration of a therapeutically effective amount of bisphosphonic acid compound in combination with lycopene may be carried out in order to treat any disorder, condition or disease for which such a compound is generally indicated. Such disorders, conditions and diseases include, for example, disturbances involving calcium or phosphate metabolism, e.g., involving bone resorption, particularly osteoporosis, Paget's disease, periprosthetic bone loss or osteolysis, malignant hypercalcemia, metastatic bone disease, multiple myeloma, periodontal disease, and tooth loss. Consequently, the use of the said pharmaceutical formulations for treating these conditions are additional aspects of the invention.

The terms“effective amount” or “pharmaceutically effective amount” refer to a nontoxic but sufficient amount of the agents to provide the desired biological result. That result can be reduction and/or alleviation of the signs, symptoms, or causes of a disease, or any other desired alteration of a biological system. For example, an “effective amount” for therapeutic uses is the amount of the composition comprising an active compound herein required to provide a clinically significant increase in healing rates in fracture repair; reversal of bone loss in osteoporosis; reversal of cartilage defects or disorders; prevention or delay of onset of osteoporosis; stimulation and/or augmentation of bone formation in fracture non-unions and distraction osteogenesis; increase and/or acceleration of bone growth into prosthetic devices; and repair of dental defects. An appropriate “effective” amount in any individual case may be determined by one of ordinary skill in the art using routine experimentation.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

The following procedure is used throughout the examples below to dissolve the active principle in the solvent system which is then encapsulated in the softgel.

Solutions of bisphosphonates embodying this invention may be prepared by first micronizing the bisphosphonate to an average particle size no greater than 80 microns, then screening the drug through a 175 mesh screen to eliminate excessively large particles, and then dissolving measured amounts of the micronized bisphosphonate in combination with the lycopene in any of the solvents specified above and in the Table Examples below.

Accordingly, the PEG 400, water and the glycerin are mixed under moderate agitation, and heated to a temperature ranging from 55° C.+/−5° C. Add the PEG E-3350 and continue mixing until homogeneous. Add the micronized bisphosphonate and lycopene active ingredients and strongly mix to have a good dispersion. The mixture is then strongly agitated until a clear transparent solution is obtained. Stop the heating and keep agitating the solution until it is at room temperature. The active material solution is suitable to be encapsulated in soft gelatin capsules.

### Example 1

| COMPONENTS | AMOUNT/mg |
|---|---|
| Monosodium Alendronate | 91.40 mg |
| Lycopene | 15.0 mg |
| PEG 400 | 148.35 mg |
| Water | 20.00 mg |
| Glycerin | 13.25 mg |
| PEG E-3350 | 7.00 mg |
| Total | 295 mg |

### Example 2

| COMPONENTS | AMOUNT/mg |
|---|---|
| Monosodium Alendronate | 71.40 |
| Lycopene | 25.0 mg |
| PEG 400 | 148.35 |
| Water | 20.00 mg |
| Glycerin | 13.25 mg |
| PEG E-3350 | 7.00 |
| Total | 285 mg |

### Example 3

| COMPONENTS | AMOUNT/mg |
|---|---|
| Sodium Risedronate | 31.40 |
| Lycopene | 50 mg |
| PEG 400 | 148.35 |
| Water | 20.00 mg |
| Glycerin | 13.25 mg |
| PEG E-3350 | 7.00 |
| Total | 270 mg |

### Example 4

| COMPONENTS | AMOUNT/mg |
|---|---|
| zoledronate | 41.40 |
| Lycopene | 25.0 mg |
| PEG 400 | 148.35 |
| Water | 20.00 mg |
| Glycerin | 13.25 mg |
| PEG E-3350 | 7.00 |
| Total | 255 mg |

### Example 5

| COMPONENTS | AMOUNT/mg |
|---|---|
| neridronate | 51.40 |
| Lycopene | 15.0 mg |
| PEG 400 | 148.35 |
| Water | 20.00 mg |
| Glycerin | 13.25 mg |
| PEG E-3350 | 7.00 |
| Total | 255 mg |

### Example 6

| COMPONENTS | AMOUNT/mg |
|---|---|
| olpadronate | 31.40 |
| Lycopene | 50.0 mg |
| PEG 400 | 148.35 |
| Water | 20.00 mg |
| Glycerin | 13.25 mg |
| PEG E-3350 | 7.00 |
| Total | 270 mg |

### Example 7

### Soft Gelatin Capsule Containing a Solubilized Composition

A soft gelatin mixture is first prepared from the following ingredients.

| INGREDIENT | WEIGHT % |
|---|---|
| Gelatin | 43.00 |
| Glycerin | 6.00 |
| Sorbitol polyol | 14.00 |
| Water | QS 100 |

The above ingredients are combined in a suitable vessel and heated with mixing at about 65° C. to form a uniform solution. Using standard encapsulation methodology, the resulting solution is used to prepare soft gelatin capsules containing approximately 280 mg of the composition as prepared in Example 1. The resulting soft gelatin capsules are suitable for oral administration.

All publications, patents, and patent applications including all cited art and bibliographic references cited herein are hereby incorporated by reference in their entirety for all purposes.

It is understood that all equivalent features are intended to be included within the claimed contents of this invention. Although the present invention has been described with reference to specific details of certain embodiments thereof, it is not intended that such detail should be regarded as limitations upon the scope of the invention, except as and to the extent that they are included in the accompanying claims.

What is being claimed is:

1. A pharmaceutical composition comprising:
(a) effective amounts of a bone resorption inhibitor; and
(b) effective amounts of lycopene.

2. A pharmaceutical formulation suitable for filling softgel capsules comprising:
(a) a therapeutically effective amount of a bone resorption inhibitor;
(b) a therapeutically effective amount of lycopene; and
(c) a solvent system comprising 50% to 85% by weight a polyethylene glycol; 5% to 15% by weight of glycerin and 5% to 20% by weight water.

3. The pharmaceutical formulation of claim 2 wherein said bone resorption inhibitor is a bisphosphonate.

4. The pharmaceutical formulation of claim 2 wherein said polyethylene glycol is a mixture of a polyethylene glycol having an average molecular weight of 400 and a polyethylene glycol having an average molecular weight of 3400.

5. The pharmaceutical formulation of claim 3 wherein said bisphosphonate is selected from the group consisting of 4-amino-1-hydroxybutylidene-1,1-bisphosphonic acid (alendronate); N,N-dimethyl-3-amino-1-hydroxypropylidene-1,1-bisphosphonic acid (mildronate, olpadronate); 1-hydroxy-3-(N-methyl-N-pentylamino)propylidene-1,1-bis-phosphonic acid (ibandronate); 1-hydroxy-2-(3-pyridyl)ethylidene-1,1-bisphosphonic acid(risedronate); 1-hydroxyethylidene-1,1-bisphosphonic acid (etidronate); 1-hydroxy-3-(1-pyrrolidinyl)propylidene-1,1-bisphosphonic acid; 1-hydroxy-2-(1-imidazolyl) etylidene-1,1-bisphosphonic acid (zoledronate); 1-hydroxy-2-(imidazo[1,2-a]pyridin-3-yl)ethylidene-1,1-bisphosphonic acid (minodronate); 1-(4-chlorophenylthio)methylidene-1,1-bisphosphonic acid (tiludronate); 1-(cycloheptylamino)methylidene-1,1-bisphosphonic acid (cimadronate, incadronate); and 6-amino-1-hydroxyhexylidene-1,1-bisphosphonic acid (neridronate), and pharmaceutically acceptable salts thereof.

6. The pharmaceutical formulation of claim 5 wherein said bisphosphonate is 4-amino-1-hydroxybutylidene-1,1-bisphosphonic acid (alendronate) or pharmaceutically acceptable salts thereof.

7. A pharmaceutical soft gelatin capsule in unit dosage form with a filling comprising a therapeutically effective amount of a bisphosphonate; a therapeutically effective amount of lycopene; and a solvent system comprising 50% to 85% by weight a polyethylene glycol; 5% to 15% by weight of glycerin and 5% to 20% by weight water.

8. The pharmaceutical soft gelatin capsule of claim 7 wherein said bisphosphonate is selected from the group consisting of 4-amino-1-hydroxybutylidene-1,1-bisphosphonic acid (alendronate); N,N-dimethyl-3-amino-1-hydroxypropylidene-1,1-bisphosphonic acid (mildronate, olpadronate); 1-hydroxy-3-(N-methyl-N-pentylamino)propylidene-1,1-bis-phosphonic acid (ibandronate); 1-hydroxy-2-(3-pyridyl)ethylidene-1,1-bisphosphonic acid(risedronate); 1-hydroxyethylidene-1,1-bisphosphonic acid (etidronate); 1-hydroxy-3-(1-pyrrolidinyl)propylidene-1,1-bisphosphonic acid; 1-hydroxy-2-(1-imidazolyl)etylidene-1,1-bisphosphonic acid (zoledronate); 1-hydroxy-2-(imidazo[1,2-a]pyridin-3-yl)ethylidene-1,1-bisphosphonic acid (minodronate); 1-(4-chlorophenylthio)methylidene-1,1-bisphosphonic acid (tiludronate); 1-(cycloheptylamino)methylidene-1,1-bisphosphonic acid (cimadronate, incadronate); and 6-amino-1-hydroxyhexylidene-1,1-bisphosphonic acid (neridronate), and pharmaceutically acceptable salts thereof.

9. The pharmaceutical soft gelatin capsule of claim 8 wherein said bisphosphonate is 4-amino-1-hydroxybutylidene-1,1-bisphosphonic acid (alendronate) or pharmaceutically acceptable salts thereof.
